(19) United States
(12) Patent Application Publication
(10) Pub. No.: US 2006/0183126 A1
(43) Pub. Date: Aug. 17, 2006

(54) Method for the detection of biomolecules based on the replication of bound nucleic acids

(75) Inventor: Sven Bulow, Hamburg (DE)

Correspondence Address: RANKIN, HILL, PORTER & CLARK LLP, 4080 ERIE STREET, WILLOUGHBY, OH, 44094-7836, US

(73) Assignee: EPPENDORG AG, Hamburg (DE)

(21) Appl. No.: 10/564,562

(22) PCT Filed: Jul. 7, 2004
(86) PCT No.: PCT/EP04/07434

§ 371 Date: Jan. 13, 2006

(30) Foreign Application Priority Data
Jul. 18, 2003 (DE) 103 32 620.0

(51) Int. Cl. C12Q 1/68; C12M 1/34

(52) U.S. Cl. 435/6; 435/287.2

(57) ABSTRACT

A biomolecules detection method, where the biomolecules to be detected are coupled to a first substance which is part of a nucleic acid replicating device, the formed biomolecule/substance complexes being bound to the solid phase bound binding molecule specific to the particular biomolecule, and, if called for, the non-bound biomolecule/substance complexes being removed by washing, the bound biomolecule/substance complexes being incubated with high molecular weight nucleic acid molecules and mononucleotides of different species, of which at least the mononucleotides of one species are fitted with detectable markings, further being incubated with a second substance complementing the first substance coupled to the biomolecules into a functional, replicating device for high molecular weight nucleic acids that binds the high molecular weight nucleic acids, and with integration of marked mononucleotides generating replicas of the high molecular weight nucleic acid molecules that do not dissociate off it, where called for the dissolved high molecular weight nucleic acid molecules and mononucleotides which do not dissociate being removed by washing, the biomolecules to be detected being ascertained from the evidence of the marked replicas.

The present invention relates to detecting biomolecules.

To attain high sensitivity in detecting biomolecules in a biomedical test sample, bio-analytic labs frequently employ the ELISA procedure (Enzyme Linked Immuno Sorbent Assay). This procedure allows detecting biomolecules down to a minimum detection limit of 100,000 molecules (10⁻¹⁸) moles.

This procedure is too insensitive for serum analysis or gene expression research because therein the proteins or peptides to be detected are present only in much lesser quantities.

Procedures are known in the state of the art that offer substantially higher detection sensitivities. Illustratively U.S. Pat. No. 5,665,539 discloses such a procedure detecting a given biomolecules (“Immuno-PCR”). In this procedure the biomolecule is bound in site specific manner—for instance by means of deposited antibodies—to a matrix. Thereupon the matrix is jointly incubated with an antibody specific to the looked for biomolecule, the non-bound antigens being removed in a washing stage.

The bound antibodies are marked by a nucleic acid molecule which is multiplied by polymerase chain reaction (PCR) and of which the amplification products first are dissolved and then are detected by agarose gel electrophoresis.

This procedure makes use of the extraordinarily high specificity of PCR and of good quantitative reproducibility. As a result the detection limit of biomolecules under appropriate conditions is better by three to five orders of magnitude than for the ELISA procedure.

However this procedure lacks specificity between the protein to be detected and the amplified nucleic acid marker. Therefore this procedure is suitable only for high-sensitivity detection of a single type of biomolecule to be detected.

A similar procedure is disclosed in U.S. Pat. No. 6,531,283 wherein the bound antibodies are marked with a nucleic acid molecule that is multiplied by means of polymerase chain reaction (RCA). Therein the amplification products are appended in the sense of a chain extension to the nucleic acid molecules, resulting in a concatenation exhibiting many repetitions of the original marker sequence, said concatenation being detectable by using appropriate steps, for instance anhybridizing fluorescence-marked nucleic acid segments that are complementary to the repetitive sequence.

This procedure incurs the drawback that only a limited selection of replication enzymes may be used, for instance the DNA polymerase of bacteriophage T7. Therefore said procedure is restricted to the properties of available polymerases. Such a restriction is a large drawback in particular as regards selecting the incubation temperature. Moreover this procedure requires a circular primer (Amplification Target Cycle) that must include sequence homologies with the nucleic acid molecules bound to the antibodies.

The objective of the present invention is to create a method allowing detecting a test sample's biomolecules at very high sensitivity and specificity while circumventing the above cited disadvantages. Another objective is to create a method for preparing a marker that may be used in such a method for detecting biomolecules, and to prepare such markers.

These problems are solved by the features of claims 1 or 2, by the features of claim 26 and by those of claim 43.

According to claim 1, the biomolecule to be detected is coupled with a substance which is part of a nucleic acid replicating device,

to bind the biomolecule-substance complexes so formed is bound to the solid phase bound binding molecules specific to the particular biomolecules,

if called for to remove by washing the non-bound biomolecule-substances complexes,

to incubate the bound biomolecule-substance complexes with high-molecular weight nucleic acid molecules and mono-nucleotides of different species, of which at least the mono-nucleotides of one species are fitted with a detectable marking, also with a second substance that complements the first substance bound to the biomolecules into a functional, replicating device for high molecular weight nucleic acids, said device binding the nucleic acid molecules and under integration of marked mononucleotides generating replicas of the high molecular weight nucleic acid molecules which do not dissociate away,

if called for to remove by washing the dissolved high molecular weight nucleic acid molecules and mononucleotides,

and determining the biomolecules to be detected by the evidence of the marked replicas.

Whereas in the cases of conventional immunoassays incubating the immobilized biomolecule fitted with a marked antibody or a cascade of antibodies, whence each biomolecule to be detected can only be imparted one or a few markings, in the case of the present invention each biomolecule can be fitted with many more markings because a plurality of marked mononucleotides is integrated into replicated nucleic acid molecule that acts as the detectable marker. Accordingly a much stronger amplified signal is generated in comparison with conventional assays. Lastly the marked biomolecules are identified by means of the specific, solid phase bound binding molecules of which the identify and position are known to the particular user.

Because of the much more amplified signal, the lowest detection limit of the method of the present invention is substantially lower than in immunoassays. However such high sensitivity requires high specificity for the amplification. In the method of the present invention, this requirement is met by the extraordinarily high specificity at which biological nucleic acid replicating device will replicate a high molecular weight nucleic acid molecule.

The method of the present invention is not restricted to a few DNA polymerases of which the very specific properties might limit said method for instance with as regards temperatures of reaction, because a plurality of nucleic acid replicating device are applicable in said method of the invention.

The method disclosed in claim 2 defines a variation as advantageous as the method of claim 1. According to claim 2, immobilized biomolecules are incubated with connection complexes consisting of molecules specifically binding to the particular biomolecules and also having a first substance which is part of a nucleic acid replicating device,

if called for removing by washing the non-bound connection complexes,

incubating the formed biomolecule connection complexes with high molecular weight nucleic acid molecules and mononucleotides of different species, of which at least the mononucleotides of one species are fitted with a detectable marking, further with a second substance which complements the first substance coupled to the biomolecules into a functional, replicating device for high molecular weight nucleic acid, said device binding the high molecular weight nucleic acid molecules and, under integration of marked mononucleotides, generates replicas of the high molecular weight nucleic acid molecules that do not dissociate off said device,

and determining the biomolecules to be detected by detecting the marked replicas.

Contrary to the case of claim 1, in claim 2 therefore it is not the biomolecules to be detected which are first coupled with the first substance into connection complexes, but instead specific binding molecules which then are incubated with the biomolecules to be detected. The biomolecules in turn were previously immobilized on a solid phase substrate.

Immobilization can be carried out for instance by non-specific adsorption or covalent binding to a suitable substrate. However, in a preferred implementation of the present invention, the biomolecules are immobilized by binding to solid phase bound specific binding molecules,

following incubation of the biomolecules with the connection complexes, the non-bound connection complexes are removed if called for by washing,

and prior to the detection of the marked replicas, the dissolved high molecular weight nucleic acid molecules and mononucleotides are removed if called for by means of washing. Incubation of the immobilized biomolecules with the connection complexes results in the known “sandwich” configuration.

In advantageous modes of implementation of the present invention, the biomolecules to be detected may be coupled covalently with the first substance, or that the binding molecules in the connection complexes are coupled covalently with the first substance.

In further modes of implementation, the biomolecules to be detected are coupled by linker systems to the first substance, or, in the connection complexes, the binding molecules are coupled by linker systems to the first substance.

Illustrative and especially preferred linker systems are the biotin-streptavidin system, the ULS platinum-linker system, the digoxigenin system or an arbitrary antigen-antibody system. However any other specifically binding system may be used. Illustratively all systems are suitable that are based on the presence of a hapten.

In one advantageous implementation of the present invention, the first substance is the β sub-unit of a DNA polymerase III and the second substance contains the remaining required sub-units of a DNA polymerase III, so that the previously cited nucleic acid replicating device is made up of these two components.

Alternatively however the first substance may be one or several sub-units of a DNA polymerase III and the second substance contains β sub-units of a DNA polymerase III as well as any further required sub-units of a DNA polymerase III.

Again and in advantageous manner, the first substance may contain β sub-units of a DNA polymerase III and the second substance may be a DNA polymerase I, the Klenow fragment of a DNA polymerase I, the Taq DNA polymerase or another DNA polymerase.

In yet another implementing mode of the present invention, the first substance is a DNA polymerase I, the Klenow fragment of a DNA polymerase I, the Taq DNA polymerase or another DNA polymerase and the second substance contains β sub-units of a DNA polymerase III.

In every one of the above modes of implementation, the actual replication of the high molecular weight nucleic acid molecule is carried out by the remaining sub-units of a DNA polymerase III, a DNA polymerase I, the Klenow fragment or the Taq DNA polymerase, whereas the β sub-unit(s) assure(s) the required high replication specificity and processability in that it (they) ensure(s) clamping the remaining sub-units or the particular enzyme to the replicating high molecular weight nucleic acid molecule. Any DNA polymerase III or its sub-units known in the field may be used. In particular sub-units of different DNA polymerases III may be used in combination.

In another preferred mode of implementation, specificity and processability are increased further in that biomolecule/substance complexes or the biomolecule/connecting complexes are incubated—in addition to the second substance—with further β sub-units of a DNA polymerase III.

The effect of the β sub-unit of a DNA polymerase III interacting with a DNA polymerase to enhance the specificity and processability of DNA replication is known for instance from U.S. Pat. No. 6,555,349 which discloses a procedure for isothermally amplifying nucleic acid molecules using a three-component polymerase consisting of a DNA polymerase (for instance DNA polymerase III), a clamp complex (for instance β sub-units) and an accessory complex (for instance γ complex). However said document only discloses its application to amplifying nucleic acid molecules. U.S. Pat. No. 6,555,349 does not describe an application relating to immunoassays or to detecting test sample molecules.

In a further advantageous implementing mode of the invention, the high molecular weight nucleic acid molecules are shaped circularly. This feature assures that neither these molecules nor the generated, marked and also circular replicas shall dissociate off the nucleic acid replicating device because the β sub-units do retain circular nucleic acid molecules but are unable to hold in place linear nucleic acid molecules.

In one advantageous implementing mode of the method of the invention, the high molecular weight nucleic acid molecules to be replicated each exhibit one sequence of replication origin against which the nucleic acid replicating device may come into contact.

In an especially advantageous implementation of the invention, the length of the high molecular weight nucleic acid molecules is at least 10 kb. In this manner a large number of marked mononucleotides are reliably integrated into the replicas and thereby strong signal amplification is assured. Compared to a PCR amplified molecule, about 20-fold more markers are integrated.

Especially preferred implementing modes provide that such markings consist of fluorescent, luminescent, radioactive or enzymatic markers. However all other suitable markers may be used.

One advantageous implementing mode of the method of the invention provides that the solid phase bound binding molecules are configured on a biochip. In this case the marked replicas are detected in especially preferred manner using biochip scanners.

In a further advantageous mode of implementation of the method of the invention, the solid phase bound binding molecules are mounted on beads. In this case the marked replicas are detected in especially preferred manner using flow detectors.

In a further advantageous implementation of the method of the invention, the solid phase bound binding molecules or the immobilized molecules are configured in a biological preparation.

Such a preparation for instance may be a histological section, a freeze fracture preparation or a Western Blot, however it may also be any other preparation comprising solid phase bound binding molecules or immobilized biomolecules.

Advantageous implementations of the method of the invention provide that preferably the biomolecules to be detected are amino acids, proteins, sugars, nucleic acids, antibodies lectins, lipids or receptors, whereas the binding molecules preferably are proteins, sugars, nucleic acids, antibodies, lectins, receptors or other specifically binding molecules.

Claim 26 defines a method for preparing a marker used to detect biomolecules, where

a first substance which is part of a nucleic acid replicating device and comprises a coupling element,

incubates with high molecular weight nucleic acid molecules and mononucleotides of different species of which at least the mononucleotides of one species comprise a detectable marking, further a second substance, further a second substance, are incubated, complementing the first substance into a functional, replicating device for high molecular weight nucleic acids, in a manner that

the device so formed shall bind the high molecular weight nucleic acids and, with integration of marked mononucleotides, shall generate replicas of the high molecular weight nucleic acid molecules, said nucleic acid molecules not dissociating off said device.

Contrary to the case of the markers used in conventional ELISA or sandwich assays, the marker prepared as above comprises many more detectable markings. Consequently a much more amplified signal is generated than with conventional assays. As a result the least detectable limit for biomolecules of a marker made by means of the method of the invention is much lower than for Elisa or sandwich assays.

In one advantageous implementing mode of this method, the coupling element is a functional group which can bond covalently with molecules to be bound.

In a further advantageous implementing mode, the coupling element is part of a linker system allowing binding the molecules to be bound.

In this respect especially preferred linker systems are the biotin-streptavidin system, the ULS-platinum linker system, the digoxigenin system or an arbitrary antigen-antibody system. However any other specifically binding system also may be used. Illustratively all systems are appropriate that are based on the presence of a hapten.

In a preferred implementing mode of this method, the first substance is connected by the coupling element to a biomolecule, namely a molecule that is the object of detection.

Equally advantageously, the first substance can be connected by the coupling element to a biomolecule, namely a molecule which in fact must be detected.

For that purpose, the biomolecules to be detected preferably are amino acids, proteins, sugars, nucleic acids, antibodies, lectins, lipids or receptors, whereas the binding molecules preferably are proteins, sugars, nucleic acids, antibodies, lectins, receptors or other specifically binding molecules.

In one advantageous implementing mode of the method of the invention, the first substance is the β sub-unit of a DNA polymerase III and the second substance contains the remaining, required sub-units of a DNA polymerase III, whereby the nucleic acid replicating device cited uppermost is made up of these two components.

Alternatively the first substance may be one or several sub-units of a DNA polymerase III and the second substance may contain β sub-units of a DNA polymerase III as well as any required further sub-units of a DNA polymerase III.

Again the first substance may advantageously contain β sub-units of a DNA polymerase III and the second substance may be DNA polymerase I, the Klenow fragment of a DNA polymerase I, the Taq DNA polymerase or another DNA polymerase.

In still another implementing mode, the first substance may be a DNA polymerase I, the Klenow fragment of a DNA polymerase I, the Taq DNA polymerase or another DNA polymerase, and the second may contain β sub-units of a DNA polymerase III.

In each of the above implementing modes of the method of the invention, the actual replication of the high molecular weight nucleic acid molecule is carried out by means of the remaining sub-units of a DNA polymerase III, by a DNA polymerase I, by the Klenow fragment or the Taq DNA polymerase, whereas the β sub-unit(s) assure(s) the required, high specificity and processability of replication by clamping the residual sub-units or the particular enzymatic to the high molecular weight nucleic acid molecule to be replicated.

Specificity and processability may be enhanced further by incubating the biomolecule/substance complexes or the biomolecule connection complexes with further β sub-units of a DNA polymerase III in addition to the second substance.

In a further advantageous implementing mode of the method of the invention, the high molecular weight nucleic acid molecules to be replicated are circular in shape. As a result, neither these replications nor those generated and marked and also circular shall dissociate off the nucleic acid replicating device because the β sub-units hold in place circular nucleic acid molecules while being unable to keep in place linear nucleic acid molecules.

In a further advantageous implementing mode of the method of the invention, the high molecular weight nucleic acids to be replicated each are fitted with one replication origin sequence against which may be set the nucleic acid replicating device.

In an especially advantageous implementing mode of the method of the invention, the high molecular weight nucleic acid molecules exhibit a length of at least 10 kb. In this manner a large number of marked mononucleotides is reliably integrated into the replicas and high signal amplification is attained. Accordingly, in comparison with a PCR amplified molecule, a 20-fold larger number of detected markers has been integrated.

In especially advantageous implementing modes, these markings consist of fluorescent, luminescent, radioactive or enzymatic markers. However all other suitable markers may be used.

According to claim 43, a biomolecules detecting marker is defined that was prepared by means of one of the above discussed.

1. A method detecting biomolecules, where
(a) the biomolecules to be detected are coupled to a first substance which is part of a nucleic acid replication device,
(b) the formed biomolecules/substance complexes are bound to the solid phase bound binding molecules that are specific for the particular biomolecules,
(c) if called for the non-bound biomolecules/substance complexes are removed by washing,
(d) the bound biomolecules/substance complexes are incubated with high molecular weight nucleic acid molecules and mononucleotides of various species of which at least the nucleotides of one species are fitted with a detectable marking, further with a second substance which complements the first substance into a functional replicating device for high molecular weight nucleic acids, said device binding the high molecular weight nuclear acid molecules and under integration of marked mononucleotides generating replicas of the high molecular weight nucleic acid molecules and mononucleotides that do not dissociate off it,
(e) if called for, removing by washing the dissolved high molecular weight nucleic acid molecules and mononucleotides,
(f) and determining the biomolecules to be detected from the detection of the marked replicas.

2. A method detecting biomolecules, where
(a) immobilized biomolecules are incubated with connecting complexes consisting of binding molecules specific to the particular biomolecules and of a first substance that is part of a nucleic acid replication device,
(b) if called for removing by washing the non-bound connection complexes,
(c) incubating the formed biomolecule/complexes with high molecular weight nucleic acid molecules and mononucleotides of various species, at least the mononucleotides of one species being fitted with a detectable marking and with a second substance that complements the first substance coupled to the biomolecules into a functional, replicating device for high molecular weight nucleic acids, said device binding the high molecular weight nucleic acid molecules and generating under integration of marked mononucleotides replicas of the high molecular weight nucleic acid molecules which do not dissociate off it,
(d) if called for removing by washing the dissolved nucleic acid molecules and mononucleotides,
(e) and determining the biomolecules to be detected by detecting the marked replicas.

3. Method for detecting biomolecules as claimed in claim 2, characterized in that
(a) prior to incubation with the connection complexes, the biomolecules are immobilized by being bound to solid phase bound specific binding molecules,
(b) following incubation of the biomolecules with the connection complexes, the non-bound connection complexes are removed by washing with water if called for,
(c) prior to detecting the marked replicas, the dissolved high molecular weight nucleic acid molecules and mononucleotides are removed by washing if called for.

4. Method for detecting biomolecules as claimed in claim 1, characterized in that the biomolecules to be detected are covalently coupled to the first substance.

5. Method for detecting biomolecules as claimed in claim 2, characterized in that, in the connection complexes, the binding molecules are covalently coupled to the first substance.

6. Method for detecting biomolecules as claimed in claim 1, characterized in that the biomolecules to be detected are coupled by linker systems to the first substance.

7. Method for detecting biomolecules as claimed in claim 2, characterized in that, in the connection complexes, the binding molecules are coupled by means of linker system to the first substance.

8. (canceled)

9. Method for detecting biomolecules as claimed in claim 1, characterized in that the first substance contains the β sub-unit of a DNA polymerase III and the second substance contains the remaining, required sub-units of a DNA polymerase III.

10. Method for detecting biomolecules as claimed in claim 1, characterized the first substance is one or several sub-units of a DNA polymerase III and the second substance contains β sub-units of a DNA polymerase III and, where called for, further required sub-units of a DNA polymerase III.

11. Method for detecting biomolecules as claimed in claim 1, characterized in that the first substance contains β sub-units of a DNA polymerase III and the second substance is a DNA polymerase I, the Klenow fragment of a DNA polymerase I, the Taq DNA polymerase or another DNA polymerase.

12. Method for detecting biomolecules as claimed in claim 1, characterized in that the first substance is a DNA polymerase I, the Klenow fragment of a DNA polymerase I, the Taq DNA polymerase or another DNA polymerase and the second substance contains β subunits of DNA polmerase III.

13-25. (canceled)

26. A method for preparing a marker for the purpose of detecting biomolecules, where
a first substance which is part of a nucleic acid replicating device and which comprises a coupling element,
is incubated with high molecular weight nucleic acid molecules and mononucleotides of different species, of which at least the mononucleotides of one species are fitted with a detectable marking, also with a second substance that complements the first substance into a functional replicating device for high molecular weight nucleic acids, in such manner that
the device so formed binds the high molecular weight nucleic acid molecules, and, while integrating marked mononucleotides, does generate replicas of the high molecular weight nucleic acid molecules that do not dissociate off it.

27. Method for preparing a marker for the purpose of detecting biomolecules as claimed in claim 26, characterized in that the coupling element is a functional group able to covalently bind with molecules to be bound.

28. Method for preparing a marker for the purpose of detecting biomolecules as claimed in claim 26, characterized in that the coupling element is part of a linker system allowing to bind the molecules to be bound.

29. (canceled)

30. Method for preparing a marker for the purpose of detecting biomolecules as claimed in claim 26, characterized in that the first substance is connected by the coupling element to a binding molecule able to specifically binding a biomolecule.

31. Method for preparing a marker for the purpose of detecting biomolecules as claimed in claim 26, characterized in that the first substance is connected by the coupling element to a biomolecule.

32. Method for preparing a marker for the purpose of detecting biomolecules as claimed in claim 30, characterized in that the binding molecules are proteins, sugars, nucleic acids, antibodies, lectins, receptors or other specifically binding molecules.

33. Method for preparing a marker for the purpose of detecting biomolecules as claimed in claim 30, characterized in that the biomolecules to be detected are amino acids, proteins, sugars, nucleic acids, antibodies, lectins, lipids or receptors.

34. Method for preparing a marker for the purpose of detecting biomolecules as claimed in claim 26, characterized in that the first substance is the β sub-unit of a DNA polymerase and the second substance contains the remaining, required sub-units of a DNA polymerase III.

35. Method for preparing a marker for the purpose of detecting biomolecules as claimed in claim 26, characterized in that the first substance is one or more sub-units of a DNA polymerase III and the second substance contains β sub-units of a DNA polymerase and any further required sub-units of a DNA polymerase III.

36. Method for preparing a marker for the purpose of detecting biomolecules as claimed in claim 26, characterized in that the first substance contains β sub-units of a DNA polymerase III and the second substance is a DNA polymerase I, the Klenow fragment of a DNA polymerase I, the Taq DNA polymerase or another DNA polymerase.

37. Method for preparing a marker for the purpose of detecting biomolecules as claimed in claim 26, characterized in that the first substance is a DNA polymerase I, the Klenow fragment of a DNA polymerase I, the Taq DNA polymerase or another DNA polymerase and the second substance contains β sub-units of a DNA polymerase III.

38-42. (canceled)

43. A marker detecting biomolecules, characterized in that it is prepared by a method defined in claim 26.

44. Method for detecting biomolecules as claimed in claim 2, characterized in that the first substance contains the β sub-unit of a DNA polymerase III and the second substance contains the remaining, required sub-units of a DNA polymerase III.

45. Method for detecting biomolecules as claimed in claim 2, characterized the first substance is one or several sub-units of a DNA polymerase III and the second substance contains β sub-units of a DNA polymerase III and, where called for, further required sub-units of a DNA polymerase III.

46. Method for detecting biomolecules as claimed in claim 2, characterized in that the first substance contains β sub-units of a DNA polymerase III and the second substance is a DNA polymerase I, the Klenow fragment of a DNA polymerase I, the Taq DNA polymerase or another DNA polymerase.

47. Method for detecting biomolecules as claimed in claim 2, characterized in that the first substance is a DNA polymerase I, the Klenow fragment of a DNA polymerase I, the Taq DNA polymerase or another DNA polymerase and the second substance contains β sub-units of DNA polmerase III.
